(19) United States
(12) Patent Application Publication
(10) Pub. No.: US 2012/0208787 A1
(43) Pub. Date: Aug. 16, 2012

(54) CRYSTALLINE FORM OF FOSAMPRENAVIR CALCIUM

(75) Inventors: Satish Manohar Bhoge, Ahmed Nagar (IN); Prakash Kshirsagar, Pune (IN); Santosh Richhariya, Sagar (IN); Kaptan Singh, Ghaziabad (IN)

(73) Assignee: RANBAXY LABORATORIES LIMITED, New Delhi, Delhi (IN)

(21) Appl. No.: 13/379,926

(22) PCT Filed: Jun. 29, 2010
(86) PCT No.: PCT/IB10/52974

§ 371 Date: Apr. 13, 2012

(30) Foreign Application Priority Data
Jun. 30, 2009 (IN) 1348/DEL/2009

(51) Int. Cl. A61K 31/665; A61P 31/18; C07F 9/655

(52) U.S. Cl. 514/99; 549/222

(57) ABSTRACT

The present invention relates to a crystalline form of fosamprenavir calcium. The crystalline form of the present invention is designated as Form II of fosamprenavir calcium. The present invention also relates to a process for the preparation of crystalline Form II of fosamprenavir calcium. The present invention further relates to a pharmaceutical composition comprising crystalline Form II of fosamprenavir calcium. The present invention relates further to a method of treating a HIV infection using crystalline Form II of fosamprenariv calium.

### FIELD OF THE INVENTION

The present invention relates to a crystalline form of fosamprenavir calcium. The crystalline form of the present invention is designated as Form II of fosamprenavir calcium. The present invention also relates to a process for the preparation of crystalline Form II of fosamprenavir calcium. The present invention further relates to a pharmaceutical composition comprising crystalline Form II of fosamprenavir calcium.

### BACKGROUND OF THE INVENTION

Fosamprenavir calcium is chemically (3S)-tetrahydrofuran-3-yl-(1S,2R)-3-[[(4-aminophenyl)sulfonyl](isobutyl)amino]-1-benzyl-2-(phosphonooxy)propylcarbamate monocalcium salt of Formula I:

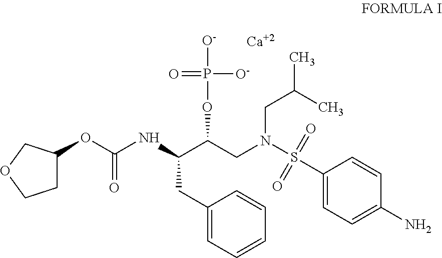

Fosamprenavir calcium is a prodrug of amprenavir, an inhibitor of HIV protease, and indicated in combination with other antiretroviral agents for the treatment of human immunodeficiency virus (HIV-1) infection.

U.S. Pat. No. 6,514,953 says that a range of salts of fosamprenavir were made including di-sodium, di-potassium, magnesium, zinc, ethylene diamine, piperazine and of these, the piperazine salt was a crystalline solid, but had the practical disadvantage of likely toxicity at the anticipated dose. U.S. Pat. No. 6,514,953 further says that the calcium salt, calcium(3S)tetrahydro-3-furanyl(15,2R)-3-[[(4-aminophenyl) sulfonyl](isobutyl)amino]-1-benzyl-2-phosphonooxy)propylcarbamate, was surprisingly found to have a stable crystalline form. U.S. Pat. No. 6,514,953 also describes processes for the preparation of fosamprenavir calcium as white microcrystalline needles using industrial methylated spirit and water. U.S. Pat. No. 6,514,953 further provides X-ray Powder Diffraction pattern of fosamprenavir calcium referred as solid state Form I.

### SUMMARY OF THE INVENTION

The present inventors have prepared a crystalline form of fosamprenavir calcium, which is significantly different from the crystalline form described in the prior art. The crystalline form of the present invention is designated as Form II of fosamprenavir calcium. Crystalline Form II of fosamprenavir calcium is stable, reproducible and suitable for developing pharmaceutical dosage forms.

### BRIEF DESCRIPTION OF THE DRAWINGS

FIG. 1 depicts the X-ray Powder Diffraction (XRPD) pattern of crystalline Form I of fosamprenavir calcium.

FIG. 2 depicts the Fourier-Transform Infra-Red (FTIR) spectrum of crystalline Form I of fosamprenavir calcium.

FIG. 3 depicts the Differential Scanning calorimetry (DSC) thermogram of crystalline Form I of fosamprenavir calcium.

FIG. 4 depicts the XRPD pattern of crystalline Form II of fosamprenavir calcium.

FIG. 5 depicts the FTIR spectrum of crystalline Form II of fosamprenavir calcium.

FIG. 6 depicts the DSC thermogram of crystalline Form II of fosamprenavir calcium.

FIG. 7 depicts the XRPD pattern of a mixture of crystalline Form I and Form II of fosamprenavir calcium.

### DETAILED DESCRIPTION OF THE INVENTION

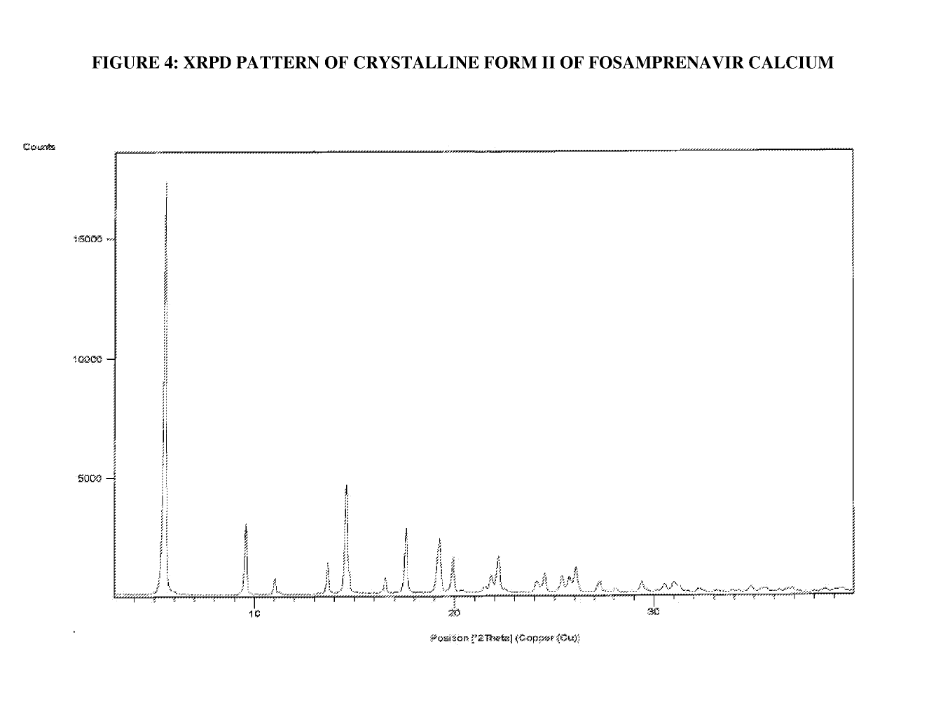

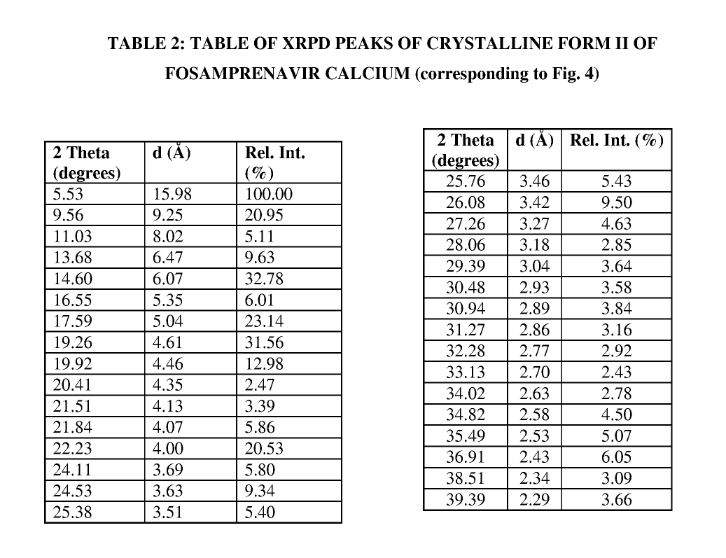

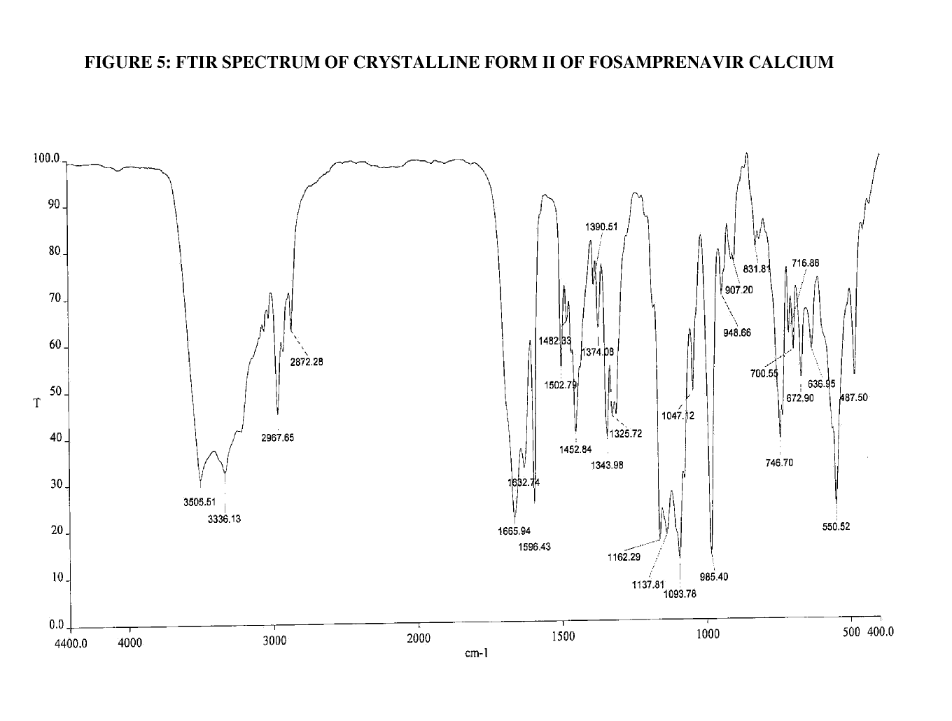

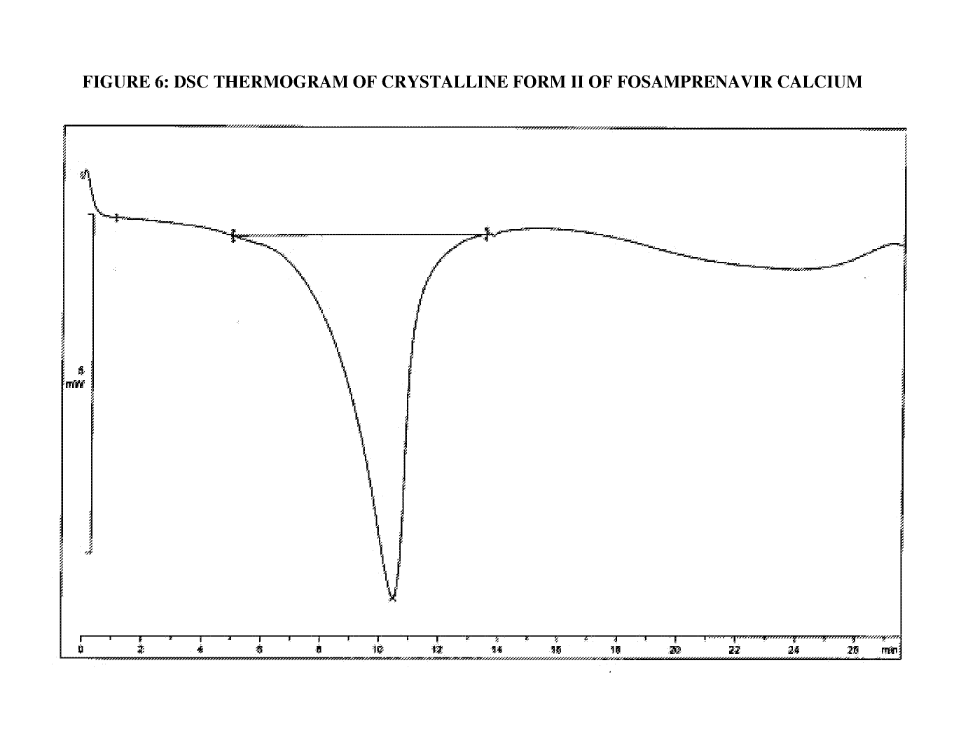

A first aspect of present invention provides crystalline Form II of fosamprenavir calcium. The crystalline Form II of fosamprenavir calcium has substantially the same XRPD (X-ray Powder Diffraction) pattern as depicted in FIG. 4. The crystalline Form II of fosamprenavir calcium is characterized by an XRPD pattern comprising interplanar spacing (d) values substantially at 15.98, 9.25, 8.02, 6.07, 5.35, 5.04, 2.86 and 2.70 (Å). The crystalline Form II of fosamprenavir calcium is further characterized by an XRPD pattern comprising interplanar spacing (d) values substantially at 15.98, 9.25, 8.02, 6.47, 6.07, 5.35, 5.04, 4.61, 4.46, 4.35, 4.13, 4.07, 4.00, 3.69, 3.63, 3.51, 3.46, 3.42, 3.27, 3.18, 3.04, 2.93, 2.89, 2.86, 2.77, 2.70, 2.63, 2.58, 2.53, 2.43, 2.34 and 2.29 (Å). The crystalline Form II of fosamprenavir calcium has substantially the same FTIR pattern as depicted in FIG. 5. The crystalline Form II of fosamprenavir calcium has substantially the same DSC thermogram as depicted in FIG. 6. The DSC thermogram of crystalline Form II of fosamprenavir calcium exhibits an endothermic peak at about 125° C. to about 135° C.

A second aspect of the present invention provides a process for the preparation of crystalline Form II of fosamprenavir calcium, wherein the process comprises:
a) dissolving fosamprenavir calcium in a water-miscible organic solvent, wherein the water-miscible organic solvent comprises a propanol;
b) treating the solution obtained in step a) with water; and
c) isolating crystalline Form II of fosamprenavir calcium from the mixture thereof.

Fosamprenavir calcium used as a starting material may be prepared according to the methods provided in the prior art, for example, U.S. Pat. No. 6,514,953. Fosamprenavir calcium is dissolved in a water-miscible organic solvent, wherein the water-miscible organic solvent comprises a propanol, for example, isopropanol or n-propanol, or a mixture thereof. The water-miscible-organic solvent may further comprise methanol, ethanol, or a mixture thereof. The dissolution may be effected by heating the mixture, for example, to a temperature of about 60° C. to about 95° C. The solution so obtained may optionally be cooled, followed by the treating with water. The mixture so obtained may be stirred at about 0° C. to about 40° C., for example, about 20° C. to about 30° C. The stirring may be carried out for about 10 minutes to about 100 hours, for example, about 5 hours to about 20 hours. The crystalline Form II of fosamprenavir calcium may be isolated from the mixture by filtration, decantation, concentration or a combination thereof.

A third aspect of the present invention provides a pharmaceutical composition comprising crystalline Form II of fosamprenavir calcium and a pharmaceutically acceptable carrier.

A fourth aspect of the present invention provides a method of treating HIV infection comprising a step of administering to a patient in need thereof a therapeutically effective amount of crystalline Form II of fosamprenavir calcium.

XRPD patterns of the samples were recorded using Panalytical X′Pert Pro X-Ray Powder Diffractometer in the range 3-40° 2θ and under tube voltage and current of 45 Kv and 40 mA respectively. Copper radiation of wavelength 1.54 Å and Xceletor detector was used.

FTIR spectra of the samples were recorded using Perkin Elmer Spectrum One instrument as potassium bromide pellets, according to the USP 25, general test methods, page 1920.

DSC thermograms were recorded using a Mettler DSC 821 instrument. About 3 to 5 mg of sample was scanned from 25° C. to 350° C. at a heating rate of 10° C./min under nitrogen flow of 50 ml/min using alumina crucibles covered with lid having one hole.

While the present invention has been described in terms of its specific embodiments, certain modifications and equivalents will be apparent to those skilled in the art and are intended to be included within the scope of the present invention.

### EXAMPLE 1

### Preparation of Crystalline Form II of Fosamprenavir Calcium

Fosamprenavir calcium (5 g) was added to isopropanol (100 ml) at 25° C. to 30° C. The temperature was raised to 80° C. to 85° C. Methanol (25 ml) was added slowly into the suspension so obtained at 80° C. to 85° C. in 10 minutes to obtain a clear solution. The solution was cooled to 70° C. to 75° C. and de-ionized water (20 ml) was added in 10 minutes at 70° C. to 75° C. The resultant mixture was slowly cooled to 25° C. to 30° C. in 2 to 2.5 hours and stirred at 25° C. to 30° C. for 10 hours to 15 hours. The solid was filtered, washed with de-ionized water (10 ml) at 25° C. to 30° C. and dried at 45° C. for 15 hours to obtain the title compound having XRPD, FTIR and DSC data as depicted in FIGS. 4, 5 and 6 respectively.

Yield: 4.5 g

Purity: 99.64%

### Example 2

### Preparation of Crystalline Form I of Fosamprenavir Calcium

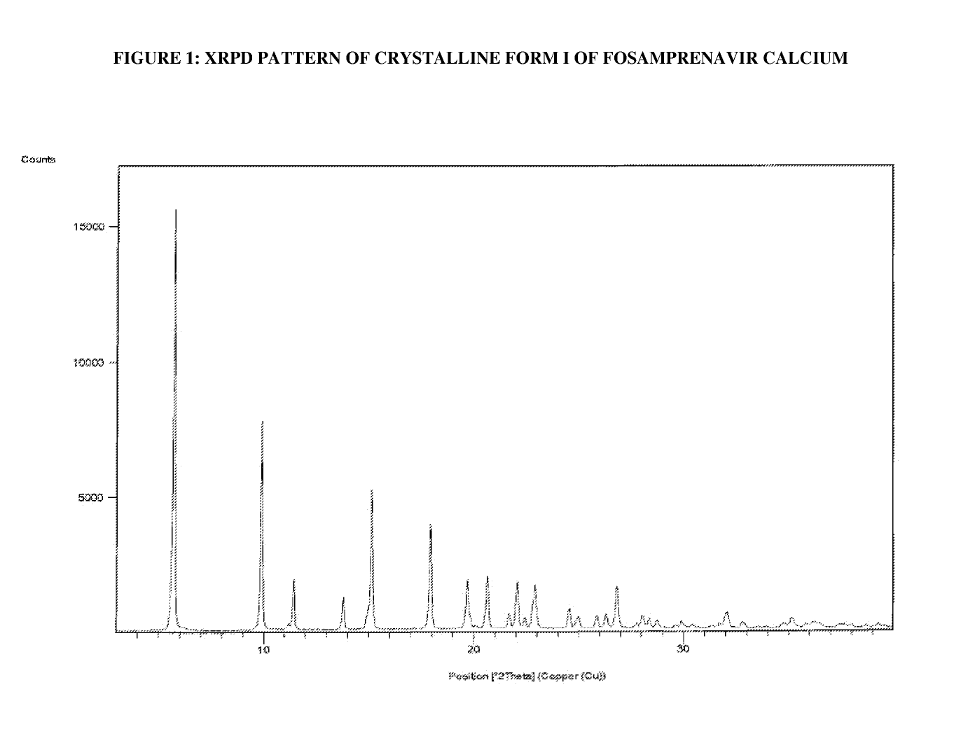

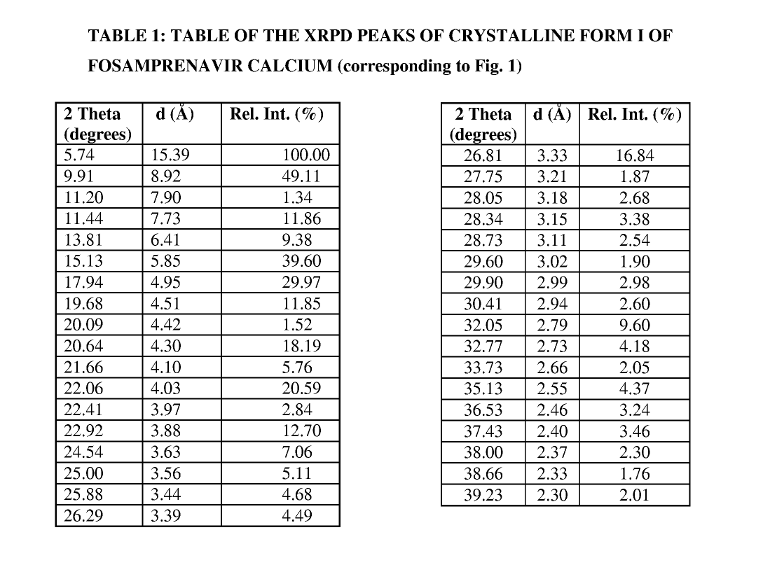

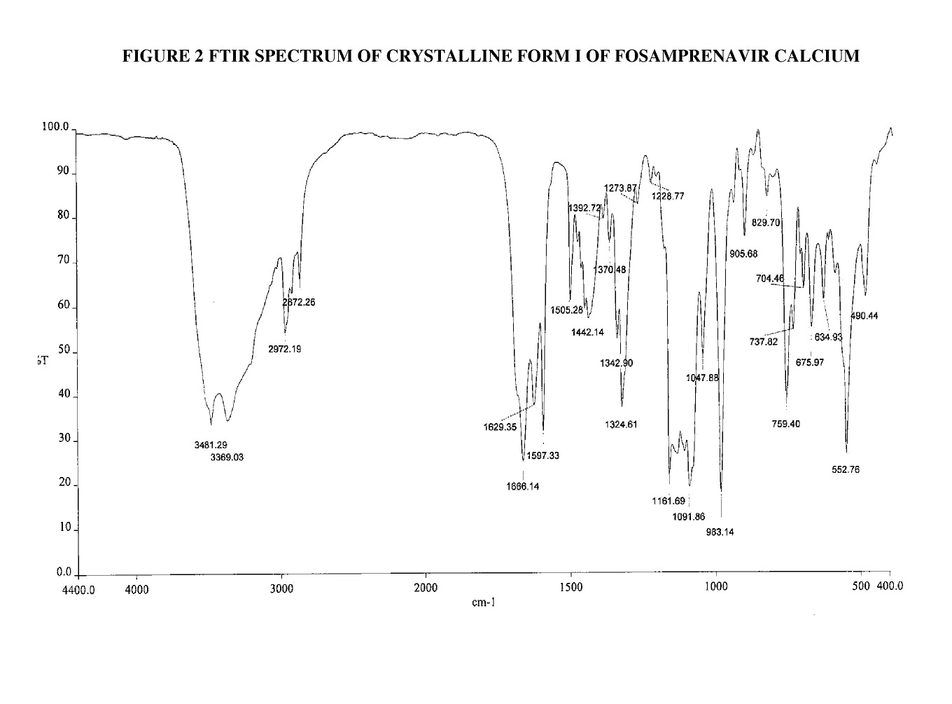

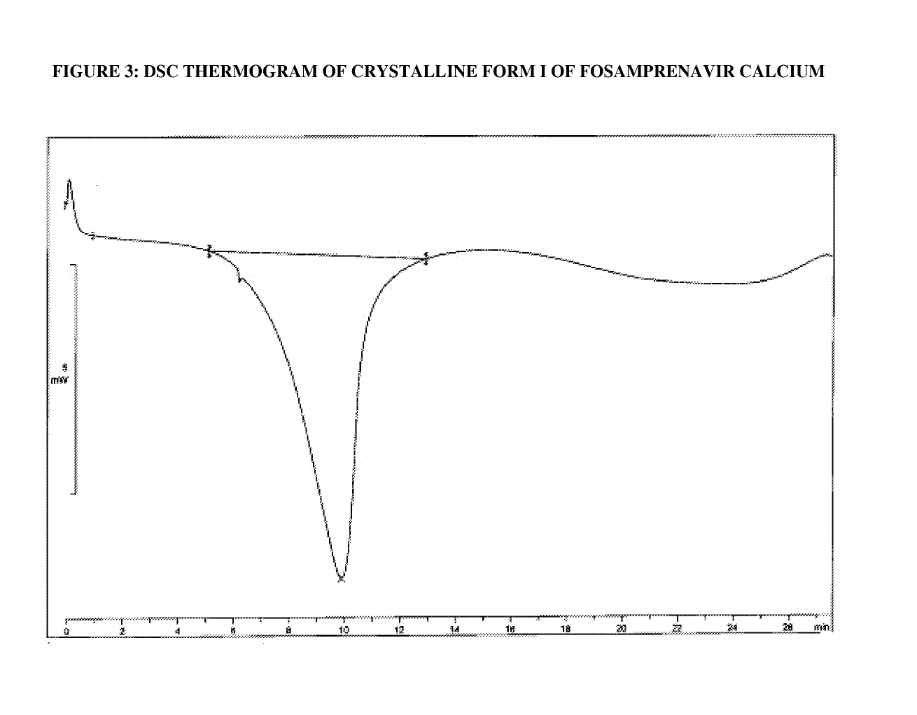

Fosamprenavir calcium (5 g) was suspended in 95:5 mixture of ethanol and methanol (75 ml) and heated to 70° C. The mixture was filtered through a Celite bed and washed with 95:5 mixture of ethanol and methanol (25 ml). The filtrate was reheated to 70° C. and water (15 ml) was added. The resulting suspension was slowly cooled to 25° C. and stirred for 3 hours at 25° C. The product was filtered, washed with a mixture of industrial methylated spirit (10 ml) and water (10 ml) and dried under vacuum at 45° C. to the constant weight to obtain the title compound having XRPD, FTIR and DSC data as depicted in FIGS. 1, 2 and 3 respectively.

Yield: 4.5 g

Purity: 99.43%

### Example 3

### Preparation of a Mixture of Form I and Form II of Fosamprenavir Calcium

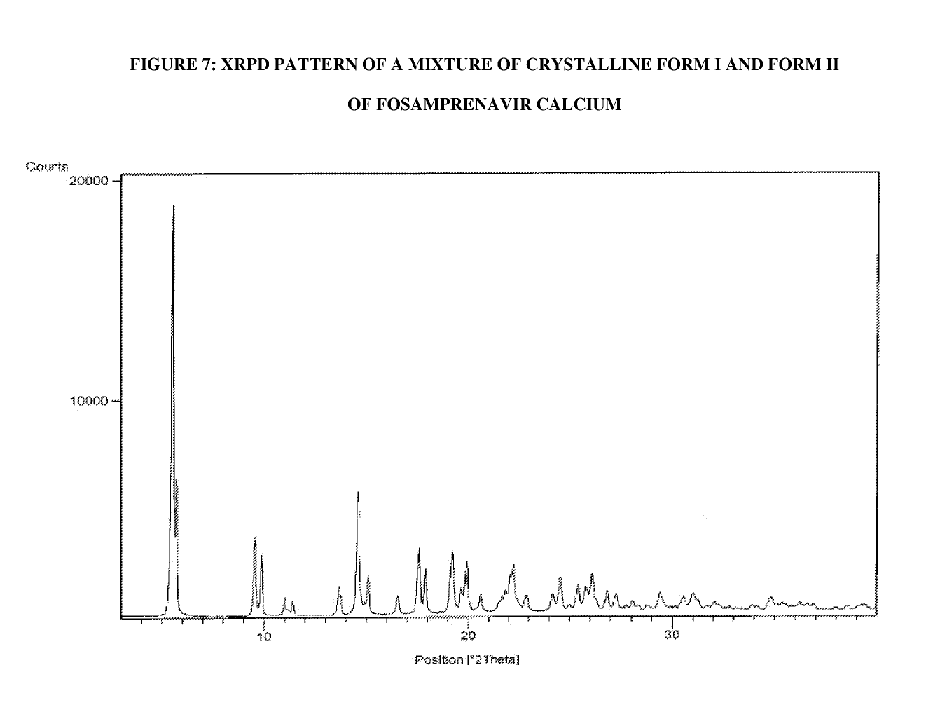

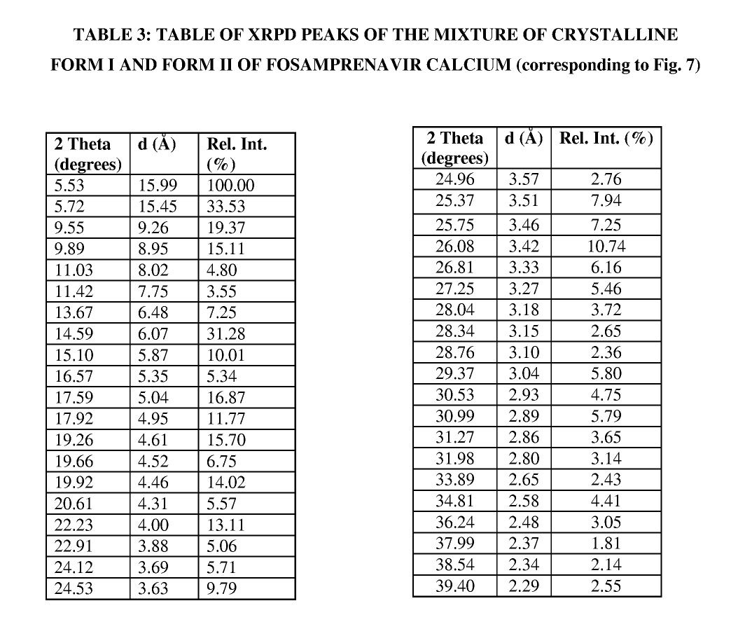

Form I of fosamprenavir calcium (1 g) was blended with Form II of fosamprenavir calcium (1 g) to obtain the title mixture having an XRPD pattern as depicted in FIG. 7.

We claim:

1. Crystalline Form II of fosamprenavir calcium characterized by an XRPD pattern comprising interplanar spacing (d) values substantially at 15.98, 9.25, 8.02, 6.07, 5.35, 5.04, 2.86 and 2.70 (Å).

2. Crystalline Form II of fosamprenavir calcium according to claim 1 characterized by an XRPD pattern comprising interplanar spacing (d) values substantially at 15.98, 9.25, 8.02, 6.47, 6.07, 5.35, 5.04, 4.61, 4.46, 4.35, 4.13, 4.07, 4.00, 3.69, 3.63, 3.51, 3.46, 3.42, 3.27, 3.18, 3.04, 2.93, 2.89, 2.86, 2.77, 2.70, 2.63, 2.58, 2.53, 2.43, 2.34 and 2.29 (Å).

3. Crystalline Form II of fosamprenavir calcium having substantially the same FTIR pattern as depicted in FIG. 5.

4. A process for the preparation of crystalline Form II of fosamprenavir calcium, wherein the process comprises:
a) dissolving fosamprenavir calcium in a water miscible organic solvent, wherein the water miscible organic solvent comprises a propanol,
b) treating the solution obtained in step a) with water and
c) isolating crystalline Form II of fosamprenavir calcium from the mixture thereof.

5. A process according to claim 4, wherein the water miscible organic solvent further comprises methanol, ethanol, or a mixture thereof.

6. A pharmaceutical composition comprising crystalline Form II of fosamprenavir calcium and a pharmaceutically acceptable carrier.

7. A method of treating HIV infection comprising a step of administering to a patient in need thereof a therapeutically effective amount of crystalline Form II of fosamprenavir calcium.
